(19) United States
(12) Patent Application Publication
(10) Pub. No.: US 2010/0261906 A1
(43) Pub. Date: Oct. 14, 2010

(54) Preparation of 6-Keto, 3-Alkoxy Morphinans

(75) Inventors: Joseph P. Haar, JR., Edwardsville, IL (US); Kevin R. Roesch, Ofallon, IL (US); David E. Gindelberger, Richmond Heights, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/757,076

(22) Filed: Apr. 9, 2010

Related U.S. Application Data
(60) Provisional application No. 61/247,015, filed on Sep. 30, 2009.
(60) Provisional application No. 61/167,876, filed on Apr. 9, 2009.

(51) Int. Cl. C07D 489/02

(52) U.S. Cl. 546/45

(57) ABSTRACT

The present invention provides processes for the preparation of saturated 6-keto, 3-alkoxy morphinans from unsaturated 6-hydroxy, 3-hydroxy morphinans, In particular, the invention provides processes that utilize catalytic isomerization and alkylation reactions for the preparation of saturated 6-keto, 3-alkoxy morphinans.

### CROSS REFERENCE TO RELATED APPLICATIONS

This application claims the benefit of U.S. Provisional Application No. 61/247,015 filed Sep. 30, 2009, and of U.S. Provisional Application No. 61/167,876 filed Apr. 9, 2009, both of which are incorporated herein in their entirety.

### FIELD OF THE INVENTION

The present invention generally relates to the preparation of saturated 6-keto, 3-alkoxy morphinans from unsaturated 6-hydroxy, 3-hydroxy morphinans. In particular, the invention relates to the use of catalytic isomerization and alkylation reactions for the preparation of saturated 6-keto, 3-alkoxy morphinans.

### BACKGROUND OF THE INVENTION

Hydrocodone is a semi-synthetic opiate with antitussive and analgesic properties that is generally used for relief of moderate to severe pain in patients where an opioid is appropriate. Studies have shown hydrocodone to be more potent than codeine, but less potent than morphine. Hydrocodone is the most frequently prescribed opiate in the United States, with nearly 130 million prescriptions for hydrocodone-containing products dispensed in 2006. A current method for the production of hydrocodone utilizes a methylation reaction to convert morphine or a mixture of morphine and codeine to codeine. The codeine then is hydrogenated to form dihydrocodeine, which is then converted to hydrocodone via an oxidation reaction. The overall process for the manufacture of hydrocodone is inefficient, however, and requires several recycling steps because of the high water solubility of codeine. Thus, there is a need for an improved process for producing hydrocodone at lower costs, with a higher yield and higher purity to meet the increasing demand for this compound.

### SUMMARY OF THE INVENTION

Among the various aspects of the present invention is the provision of processes for the preparation of a 6-keto, 3-alkoxy morphinan (such as, e.g., hydrocodone) from an unsaturated 6-hydroxy, 3-hydroxy morphinan (such as, e.g., morphine or codeine).

One aspect of the invention encompasses a process for the preparation of a 6-keto, 3-alkoxy morphinan. The process comprises (a) contacting a 6-hydroxy, 3-hydroxy morphinan with a transition metal catalyst to form a 6-keto, 3-hydroxy morphinan; and (b) contacting the 6-keto, 3-hydroxy morphinan with an alkylating agent and a proton acceptor to form the 6-keto, 3-alkoxy morphinan.

Another aspect of the invention provides a process for the preparation of a compound comprising Formula (III). The process comprises:
(a) contacting a compound comprising Formula (I) with a transition metal catalyst to form a compound comprising Formula (II); and
(b) contacting the compound comprising Formula (II) with an alkylating agent comprising R³ and a proton acceptor to form the compound comprising Formula (III):

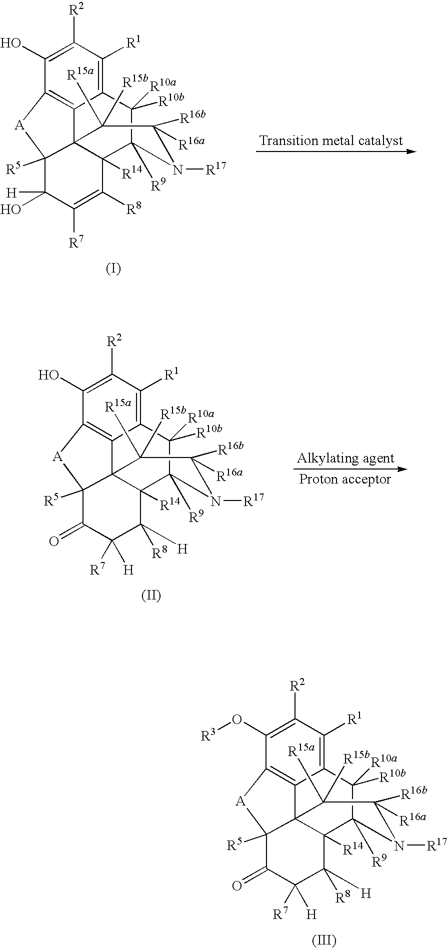

wherein:
A is a heteroatom selected the group consisting of oxygen and sulfur;
R¹ and R² are independently selected from the group consisting of hydrogen, halogen, hydroxy, protected hydroxy, {—}SH, {—}SR¹⁶¹¹, {−}OR¹⁶¹¹, and {—}NR¹⁶¹¹R¹⁶¹², hydrocarbyl, and substituted hydrocarbyl;
R³ is selected from the group consisting of hydrocarbyl and substituted hydrocarbyl;
R⁵, R⁷, R⁸, R⁹, R^{10a}, R^{10b}, R¹⁴, R^{15a}, R^{15b}, R^{16a}, R^{16b}, and R¹⁷ are independently selected from the group consisting of hydrogen, halogen, hydroxy, {—}SH, {—}SR¹⁶¹¹, {—}OR¹⁶¹¹, and {—}NR¹⁶¹¹R¹⁶¹², hydrocarbyl, and substituted hydrocarbyl; provided that any of R^{10a} and R^{10b}, R^{15a} and R^{15b}, and R^{16a} and R^{16b} may together form a moiety selected from the group consisting of {═}O, {═}S, and {═}NR¹⁶¹³;
R¹⁶¹¹, R¹⁶¹², and R¹⁶¹³ are independently selected from the group consisting of hydrocarbyl and substituted hydrocarbyl; and
one or more of R¹, R², R⁵, R⁷, R⁸, R⁹, R^{10a}, R^{10b}, R¹⁴, R^{15a}, R^{15b}, R^{16a}, and R^{16b} may form part of a ring or ring system selected from the group consisting of carbocyclic, heterocyclic, aryl, heteroaryl, and combinations thereof.

Other aspects and features of the invention are detailed below.

### DETAILED DESCRIPTION OF THE INVENTION

The present invention provides efficient processes for the preparation of saturated 6-keto, 3-alkyloxy morphinans from 6-hydroxy, 3-hydroxy morphinans. The process comprises two steps. The first step comprises a catalytic isomerization reaction that converts an unsaturated ring moiety comprising a 6-hydroxy moiety into a saturated ring moiety comprising a 6-keto moiety. The second step comprises an alkylation reaction, wherein the 3-hydroxy moiety is converted into a 3-alkoxy moiety. Each step of the reaction is efficient and produces a product of high purity. The overall process, therefore, is cost effective and high yielding.

### (I) Processes for the Preparation of 6-Keto, 3-Alkoxy Morphinans

One aspect of the present invention provides a process for the conversion of a 6-hydroxy, 3-hydroxy morphinan into a 6-keto, 3-alkoxy morphinan. More specifically, the process comprises (a) contacting an unsaturated 6-hydroxy, 3-hydroxy morphinan with a transition metal catalyst to form a saturated 6-keto, 3-hydroxy morphinan, and (b) contacting the 6-keto, 3-hydroxy morphinan with an alkylating agent and a proton acceptor to form the 6-keto, 3-alkoxy morphinan. In exemplary embodiments, the process may be used to convert morphine or a mixture of morphine and codeine to hydrocodone.

### (II) Processes for the Preparation of a Compound Comprising Formula (III)

Another aspect of the present invention encompasses a process for the preparation of a compound comprising Formula (III). The process comprises contacting a compound comprising Formula (I) with a transition metal catalyst, wherein the compound comprising Formula (I) undergoes a double bond isomerization to form a compound comprising Formula (II). The process further comprises contacting the compound comprising Formula (II) with an alkylating agent comprising R³ and a proton acceptor to form the compound comprising Formula (III). For the purpose of illustration, Reaction Scheme 1 depicts preparation of the compound comprising Formula (III) according to this aspect of the invention:

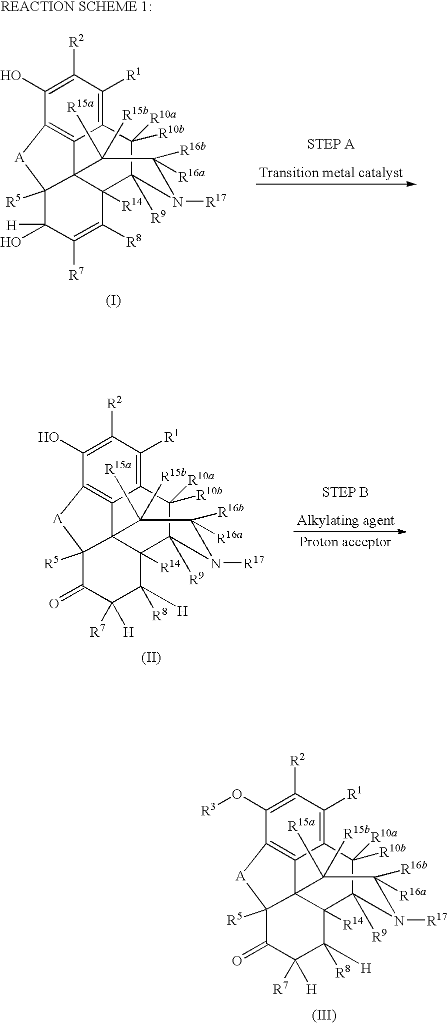

wherein:
A is a heteroatom selected the group consisting of oxygen and sulfur;
R¹ and R² are independently selected from the group consisting of hydrogen, halogen, hydroxy, protected hydroxy, {—}SH, {—}SR¹⁶¹¹, {—}OR¹⁶¹¹, and {—}NR¹⁶¹¹R¹⁶¹², hydrocarbyl, and substituted hydrocarbyl;
R³ is selected from the group consisting of hydrocarbyl and substituted hydrocarbyl;
R⁵, R⁷, R⁸, R⁹, R^{10a}, R^{10b}, R¹⁴, R^{15a}, R^{15b}, R^{16a}, R^{16b}, and R¹⁷ are independently selected from the group consisting of hydrogen, halogen, hydroxy, {—}SH, {—}SR¹⁶¹¹, {—}OR¹⁶¹¹, and {—}NR¹⁶¹¹R¹⁶¹², hydrocarbyl, and substituted hydrocarbyl; provided that any of R^{10a} and R^{10b}, R^{15a} and R^{15b}, and R^{16a} and R^{16b} may together form a moiety selected from the group consisting of {═}O, {═}S, and {═}NR¹⁶¹³;
R¹⁶¹¹, R¹⁶¹², and R¹⁶¹³ are independently selected from the group consisting of hydrocarbyl and substituted hydrocarbyl; and
one or more of R¹, R², R⁵, R⁷, R⁸, R⁹, R^{10a}, R^{10b}, R¹⁴, R^{15a}, R^{15b}, R^{16a}, and R^{16b} may form part of a ring or ring system selected from the group consisting of carbocyclic, heterocyclic, aryl, heteroaryl, and combinations thereof.

In a preferred embodiment, A is oxygen, and each of R¹, R², R⁵, R⁷, R⁸, R⁹, R^{10a}, R^{10b}, R^{15a}, R^{15b}, R^{16a}, and R^{16b} is hydrogen. In another preferred embodiment, R³ is alkyl, substituted alkyl, hydroxyalkyl, carboxyalkyl, (alkyloxycarbonyl)alkyl, aryl alkyl, alkenyl, substituted alkenyl, aryl, and substituted aryl. In a further preferred embodiment, R¹⁴ is hydrogen or hydroxy. In yet another preferred embodiment, R¹⁷ is selected from the group consisting of hydrogen, alkyl, cycloalkyl, cycloalkylmethyl, allyl, and aryl.

In an exemplary embodiment, A is oxygen; each of R¹, R², R⁵, R⁷, R⁸, R⁹, R^{10a}, R^{10b}, R^{15a}, R^{15b}, R^{16a}, and R^{16b} is hydrogen; R³ is methyl, R¹⁴ is hydrogen; and R¹⁷ is methyl.

### (a) Step A—Reaction Mixture

Step A of the process comprises contacting the compound comprising Formula (I) with a transition metal catalyst such that the allyl alcohol ring moiety of the compound comprising Formula (I) is catalytically isomerized to the saturated ketone ring moiety of the compound comprising. Formula (II). The process commences with the formation of a reaction mixture comprising the substrate, i.e., the compound comprising Formula (I), and the transition metal catalyst.

(i) Transition Metal Catalyst

A wide variety of transition metals or complexes thereof may be used to catalyze the isomerization. In general, any transition metal may be used in the process of the invention. Preferably, the transition metal will be a platinum group metal such as ruthenium, rhodium, palladium, osmium, iridium, or platinum. In exemplary embodiments, the transition metal may be ruthenium, rhodium, or palladium. A skilled artisan appreciates that the valence of the platinum group metal may vary.

In some embodiments, the transition metal catalyst may be the transition metal element itself. For example, the transition metal element may be a powder or a sponge, such as, e.g., ruthenium powder, rhodium powder, ruthenium sponge, rhodium sponge, palladium sponge, and so forth. Alternatively, the transition metal element may be rhodium black, ruthenium black, palladium black, etc. In still other embodiments, the transition metal element may be immobilized on a solid surface or support. Suitable examples include, but are not limited to, ruthenium on carbon, rhodium on carbon, palladium on carbon, ruthenium on alumina, rhodium on alumina, platinum on alumina, palladium on alumina, rhodium on silica, palladium on silica, palladium on charcoal, palladium on pumice, and so forth.

In other embodiments, the transition metal catalyst may be a transition metal salt. Non-limiting examples of suitable salts include acetates, acetyacetonates, alkoxides, butyrates, carbonyls, dioxides, halides, hexonates, hydrides, mesylates, octanates, nitrates, nitrosyl halides, nitrosyl nitrates, sulfates, sulfides, sulfonates, phosphates, trifluoromethanesulfonates, trimethylacetates, tosylates, and combinations thereof. Exemplary transition metal salts include RuCl₃, RuBr₃, Ru(CF₃SO₃)₂, RhCl₃, Rh₂(SO₄)₃, PdCl₂, and Pd(OAc)₂. The transition metal salt may be soluble (i.e., homogeneous). Alternatively, the transition metal salt may be immobilized on a solid support (i.e., heterogeneous). The transition metal salt may be immobilized on the solid support via noncovalent or covalent bonds. Suitable solid supports include silicas, alumina, titania, carbondium, zirconia, activated charcoal, zeolites, clays, polymers, ceramics, and activated carbon. Suitable silicas include silicon dioxide, amorphous silica, and microporous or mesoporous silicas. A polymer may be a natural polymer, a synthetic polymer, a semi-synthetic polymer, or a copolymer. Non-limiting examples of polymers include agarose, cellulose, nitrocellulose, methyl cellulose, polyacrylic, polyacrylamide, polyacrylonitrile, polyamide, polyether, polyester, polyethylene, polystyrene, polysulfone, polyvinyl chloride, polyvinylidene, methacrylate copolymer, and polystyrene-vinyl chloride copolymer.

In further embodiments, the transition metal catalyst may be a transition metal complex. In general, a transition metal complex comprises the transition metal and 4, 5, or 6 coordinate species with oxidation states ranging from 0 to 8. The complexes may be ionic, or the complexes may comprise covalently bound ligands and counter ions. Alternatively, the complexes may comprise a mixture of ionic and covalent bonds between the metal, ligand(s), and/or counter ion(s). The ligand may be monodentate or polydentate. Non-limiting examples of suitable ligands include arene ligands, olefin ligands, alkyne ligands, heterocycloalkyl ligands, heteroaryl ligands, alkyl ligands, cyclopentadienyl ligands, hydride ligands, amine ligands, carbonyl ligands, nitrogen donor ligands, phosphorous donor ligands, oxygen donor ligands, and so forth. The ligand may also be a solvent such as, e.g., DMSO, methanol, methylene chloride, tetrahydrofuran, acetone, ethanol, pryridine, or a tetraalkylammonia compound. Suitable counter ions include, but are not limited to, halides, BF₄, PF₆, ClO₄, CHO₂, CF₃SO₃, CH₃CO₂, ArCO₂, CH₃SO₃, p-tolylSO₃, HSO₄, H₂PO₄, and hydrocarbyl anions. Numerous transition metal complexes are detailed in “Transposition of Allylic Alcohols into Carbonyl Compounds Mediated by Transition Metal Complexes” by Uma et al., Chem. Rev. 103: 27-51 (2003). Preferred transition metal complexes include (phosphine)ₓPdCl₂, (PPh₃)₄Pd, RhCl(PPh₃)₃, and (arene)RuX₂ (e.g., the cymene dimer of ruthenium dichloride).

The transition metal complex may be soluble (i.e., homogeneous). Alternatively, the transition metal complex may be immobilized on a solid support (i.e., heterogeneous). The transition metal complex may be immobilized on the solid support via noncovalent or covalent bonds. Examples of suitable solid supports are listed above.

In still other embodiments, the transition metal catalyst may be a complex comprising the transition metal and a tertiary phosphite, a tertiary phosphine, or a tertiary phosphine halide as detailed in U.S. Pat. Nos. 7,321,038, 7,399,858, and 7,323,565, each of which is incorporated herein in its entirety. In yet another embodiment, transition metal catalyst may be a complex comprising the transition metal and an amine phosphine complex as described in U.S. Pat. No. 7,399,859, which is incorporated herein in its entirety.

In additional embodiments, the transition metal catalyst may be a bis-allyl transition metal complex. For example, the bis-allyl transition metal complex may be a bis-n³-bonded ruthenium complex such as, e.g., {Ru(η³:η³-C₁₀H₁₆)(μ-Cl)Cl}₂, Ru(η³:η²:η³-C₁₂H₁₈)Cl₂, }Ru(η³:η³-C₁₂H₁₈)(μ-Cl)Cl}₂, or bis(η³-allyl)-ruthenium-(1,5-cyclooactadiene).

In exemplary embodiments, the transition metal catalyst may be RuCl₂(PPh₃)₃, RuCl₂(PPh₃)₄, RuH₂(PPh₃)₄, RhCl(PPh₃)₃, a bis-allyl ruthenium complex, RuCl₂(dmso)₄, Pd black, Ru black, or RuCl₃.H₂O.

The weight ratio of the compound comprising Formula (I) to the transition metal catalyst can and will vary. In general, the weight ratio of the compound comprising Formula (I) to the transition metal catalyst may range from about 1:0.0001 to about 1:0.1. In various embodiments, the weight ratio of the compound comprising Formula (I) to the transition metal catalyst may range from about 1:0.0001 to about 1:0.001, from about 1:0.001 to about 1:0.01, or from about 1:0.01 to about 1:0.1. In preferred embodiments, the weight ratio of the compound comprising Formula (I) to the transition metal catalyst may range from about 1:0.002 to about 1:0.05. In an exemplary embodiment, the weight ratio of the compound comprising Formula (I) the transition metal catalyst may range form about 1:0.005 to about 1:0.02.

The pH of the reaction mixture may be adjusted to optimize activity of the transition metal catalyst. In general, the optimal pH will vary depending upon the nature of the transition metal catalyst. A person of skill in the art will know how to determine the optimal pH level for the transition metal catalyst of interest.

(ii) Solvent

In general, step A of the process of the invention is performed in the presence of a solvent. The solvent may be a protic solvent, an aprotic solvent, or combinations thereof. Non-limiting examples of suitable protic solvents include methanol, ethanol, isopropanol, n-propanol, isobutanol, n-butanol, s-butanol, t-butanol, formic acid, acetic acid, water, and combinations thereof. Examples of suitable aprotic solvents include, but are not limited to, acetone, acetonitrile, diethoxymethane, N,N-dimethylformamide (DMF), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), 1,4-dioxane, N-methyl-2-pyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyl tetrahydrofuran, trichloromethane, and combinations thereof. In preferred embodiments, the solvent may be a protic solvent. In one preferred embodiment, the solvent may be an alcohol. In another preferred embodiment, the solvent may be ethanol. In still another preferred embodiment, the solvent may be a mixture of ethanol and water.

The weight ratio of the solvent to the compound comprising Formulas (I) can and will vary. Typically, the weight ratio of the solvent to the compound comprising Formula (I) may range from about 0.5:1 to about 10:1. In various embodiments, the weight ratio of the solvent to the compound comprising Formula (I) may range from about 0.5:1 to about 2:1, from about 2:1 to about 5:1, from about 5:1 to about 10:1. In preferred embodiments, the weight ratio of the solvent to the compound comprising Formula (I) may range from about 2:1 to about 4:1.

### (b) Step A—Reaction Conditions

Typically, step A of the process of the invention is performed in one step; that is, the substrate, the catalyst, the optional proton donor, and the solvent are mixed together in a reaction vessel. The reaction is allowed to proceed at a temperature that may range from about 10° to about 120° C. In preferred embodiments, the temperature of the reaction may range from about 45° to about 100° C., or more preferably from about 65° to about 100° C. In an exemplary embodiment, the temperature of the reaction may range from about 65° to about 75° C. in another exemplary embodiment, the temperature of the reaction may range from about 80° to about 100° C. The reaction may be conducted under ambient pressure, and preferably under an inert atmosphere (e.g., nitrogen or argon).

The duration of the reaction can and will vary. For example, the reaction may be allowed to proceed for about 1 hr, 2, hr, 3, hr, 4, hr, 5, hr, 6 hr, 8 hr, 10 hr, 12 hr, 15 hr, 18 hr, or 24 hr. Typically, however, the reaction is allowed to proceed for a sufficient period of time until the reaction is complete, as determined by any method known to one skilled in the art, such as chromatography (e.g., HPLC). In this context, a “completed reaction” generally means that the reaction mixture contains a significantly diminished amount of the compound comprising Formula (I) and a significantly increased amount of the compound comprising Formula (II) compared to the amounts of each present at the beginning of the reaction. Typically, the amount of the compound comprising Formula (I) remaining in the reaction mixture may be less than about 3%, less than about 1%, and preferably less than about 0.5%.

Upon completion of the reaction, the reaction mixture may be cooled and the compound comprising Formula (II) may be isolated by distillation, phase extraction, precipitation, filtration, crystallization, or other means familiar to those of skill in the art. The final product may be washed and dried, and analyzed by HPLC, HPLC, MS, NMR, IR, or TGA. Alternatively, the compound comprising Formula (II) formed during step A may not be isolated and the mixture comprising the compound comprising Formula (II) may be used directly for step B, as detailed below. Stated another way, the entire two-step process may comprise a one-pot process.

The yield of the compound comprising Formula (II) can and will vary. Typically, the molar yield of the compound comprising Formula (II) may be at least about 60%. In preferred embodiments of the invention, the molar yield of the compound comprising Formula (II) may be at least about 65%, or at least about 70%. In exemplary embodiments, the molar yield of the compound comprising Formula (II) may be at least about 75%, at least about 80%, or at least about 85%. In other exemplary embodiments, the molar yield of the compound comprising Formula (II) may be at least about 90%, at least about 95%, at least 97%, or at least about 99%.

### (c) Step B—Reaction Mixture

Step B of the process of the invention encompasses an alkylation reaction. More specifically, the compound comprising Formula (II) is contacted with an alkylating agent and a proton acceptor to form the compound comprising Formula (III).

(i) Alkylating Agent

In step B, the reaction mixture comprises the compound comprising Formula (II) and an alkylating agent comprising R³, wherein R³ is hydrocarbyl or substituted hydrocarbyl. In preferred embodiments, R³ may be alkyl, substituted alkyl, hydroxyalkyl, carboxyalkyl, (alkyloxycarbonyl)alkyl, aryl alkyl, alkenyl, substituted alkenyl, aryl, and substituted aryl. A variety of alkylating agents are suitable for use in the process of the invention. In one embodiment, the alkylating agent may be a halide. Suitable halides include, but are not limited to, alkyl halides, hydroxyalkyl halides, aryl alkyl halides, carboxyalkyl halides, (alkyloxycarbonyl)alkyl halides, allyl halides, and vinyl halides. In another embodiment, the alkylating agent may be an oxide. Non-limiting examples of suitable oxides include alkylene oxides, aryl substituted alkylene oxides, and halogen substituted alkylene oxides. In a further embodiment, the alkylating agent may be a sulfonate. Examples of suitable sulfonates include without limit alkyl sulfonates, hydroxyalkyl sulfonates, aryl alkyl sulfonates, carboxyalkyl sulfonates, (alkyloxycarbonyl)alkyl sulfonates, allyl sulfonates, and alkyl halogen-substituted sulfonates. In yet another embodiment, the alkylating agent may be a quaternary ammonium salt. Non-limiting examples of suitable quaternary ammonium salts include trialkyl aryl ammonium salts, trialkyl alkyl ammonium salts, and tetraalkyl ammonium salts, wherein the associated anion may be halide, sulfate, or alkoxide. In still another embodiment, the alkylating agent may be an alkyl carbonate.

Suitable alkylating agents include methyl halide, ethyl halide, propyl halide, butyl halide, benzyl halide, ethylene oxide, propylene oxide, butylene oxide, epoxybutene, amylene oxide, glycidol, styrene oxide, epichlorohydrin, methyl benzenesulfonate, ethyl benzenesulfonate, methyl methanesulfonate, ethyl methanesulfonate, propyl methyl sulfonate, methyl p-toluene sulfonate, ethyl p-toluene sulfonate, methyl trifluoromethanesulfoante, methyl fluorosulfonate, ethyl fluorosulfonate, methyl chloroacetic acid, ethyl chloroacetic acid, sodium chloroacetate, chloroacetic acid, 1-N,N-dialkylamino-2-chloroethane, trialkylphenylammonium halide, trialkylphenylammonium sulfate, trialkylphenylammonium alkoxide, trialkylalkylammonium halide, tetraalkylammonium halide, dimethyl carbonate, diethylcarbonate, methyl propyl carbonate, dipropyl carbonate, and t-butyl carbonate.

In preferred embodiments, the alkylating agent may be a methylating agent (i.e., R³ is methyl). Suitable methylating agents include methyl chloride, methyl iodide, methyl bromide, trimethylphenylammonium chloride, trimethylphenylammonium bromide, trimethylphenylammonium iodide, trimethylphenylammonium sulfate, trimethylphenylammonium methoxide, trimethylphenylammonium ethoxide, trimethyldodecylammonium chloride, trimethyldodecylammonium bromide, trimethyldodecylammonium iodide, tetramethylammonium chloride, tetramethylammonium bromide, tetramethylammonium iodide, dimethyl sulfate, methyl-p-toluenesulfonate, methyl benzenesulfonate, methyl methanesulfonate, methyl trifluoromethansulfonate, dimethyl carbonate, diazomethane, 2,2-dimethyoxypropane, dimethylzine, iodomethane, methyl fluorosulfonate, trimethylsilyldiazomethane, and combinations thereof. In exemplary embodiments, the methylating agent may be trimethylphenylammonium chloride, trimethylphenylammonium bromide, and trimethylphenylammonium sulfate.

The molar ratio of the compound comprising Formula (II) to the alkylating agent can and will vary. In general, the molar ratio of the compound comprising Formula (II) to the alkylating agent will range from about 1:0.5 to about 1:5. In preferred embodiments, the molar ratio of the compound comprising Formula (II) to the alkylating agent may be about 1:0.8, about 1:0.9, about 1:1, about 1:1.1, about 1:1:2, about 1:1.3, about 1:1.4, about 1:1.5, about 1:1.6, about 1:1.7, about 1:1.8, about 1:1.9, or about 1:2. In an exemplary embodiment, the molar ratio of the compound comprising Formula (II) to the alkylating agent may be about 1:1.

(ii) Proton Acceptor

The reaction mixture of step B also comprises a proton acceptor. Suitable proton acceptor include those in which the conjugate acid of the proton acceptor has a pKb less than about 6. Non-limiting examples of suitable proton acceptors having this characteristic include alkali or alkaline earth metal hydroxides such as e.g., sodium hydroxide, potassium hydroxide, calcium hydroxide, and the like; alkali or alkaline earth metal carbonates such as e.g., potassium carbonate, sodium carbonate, lithium carbonate, and so forth; alkali or alkaline earth metal bicarbonates such as e.g., sodium bicarbonate, potassium bicarbonate, and the like; alkali metal alkoxides or aryloxides such as e.g., sodium ethoxide; potassium ethoxide, lithium ethoxide, sodium t-butoxide, potassium t-butoxide, and so forth; alkali metal alkylcarboxylates or arylcarboxylates such as sodium acetate, sodium propionate, and the like; alkali metal phosphates such as e.g., sodium di-basic phosphate, sodium tri-basic phosphate, potassium di-basic phosphate, potassium tri-basic phosphate, and so forth; alkali or alkaline earth metal amides such as e.g., sodium amide potassium amide, calcium amide, and the like; secondary amines such as dimethylamine, diethylamine, diisopropylamine, and so forth; hindered amines such as piperidine, pyrrolidine, and the like; and tertiary amines such as e.g., trimethylamine, triethylamine, and so forth. In exemplary embodiments, the proton acceptor may be sodium ethoxide or sodium hydroxide.

The molar ratio of the compound comprising Formula (II) to the proton acceptor can and will vary. In general, the molar ratio of the compound comprising Formula (II) to the proton acceptor will range from about 1:1 to about 1:20. In various embodiments, the molar ratio of the compound comprising Formula (II) to the proton acceptor may range from about 1:1 to about 1:2.5, from about 1:2.5 to about 1:5, from about 1:5 to about 1:10, or from about 1:10 to about 1:20. In an exemplary embodiment, the molar ratio of the compound comprising Formula (II) to the proton acceptor may range from about 1:5 to about 1:10, or preferably about 1:8.

(iii) Solvent

Step B of the process is generally performed in the presence of a solvent. The solvent may be an organic solvent, an aprotic solvent, a protic solvent, or combinations thereof. Non-limiting examples of suitable organic solvents include alkane and substituted alkane solvents (including cycloalkanes), aromatic hydrocarbons, esters, ethers, ketones, combinations thereof, and the like. Specific organic solvents that may be employed, include, for example, benzene, butyl acetate, t-butyl methylether, t-butyl methylketone, chlorobenzene, chloroform, chloromethane, cyclohexane, dichloromethane, dichloroethane, diethyl ether, ethyl acetate, diethylene glycol, fluorobenzene, heptane, hexane, isobutylmethylketone, isopropyl acetate, methylethylketone, methyltetrahydrofuran, pentyl acetate, n-propyl acetate, toluene, and combinations thereof. Exemplary organic solvents include diethyl ether, hexane, methyl t-butyl ether, toluene, xylene, and combinations thereof. Suitable aprotic solvents include, without limit, acetone, acetonitrile, diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), N-methyl-2-pyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyl tetrahydrofuran, trichloromethane, and combinations thereof. Exemplary aprotic solvents include acetonitrile, N,N-dimethylformamide, 1,4-dioxane, N-methyl pyrrolidone, tetrahydrofuran, and combinations thereof. Non-limiting examples of suitable protic solvents include methanol, ethanol, isopropanol, n-propanol, isobutanol, n-butanol, s-butanol, t-butanol, formic acid, acetic acid, water, and combinations thereof. Exemplary protic solvents include methanol, ethanol, and combinations thereof.

The weight ratio of the solvent to the compound comprising Formula (II) can and will vary. In general, the weight ratio of the solvent to the compound comprising Formula (II) will range from about 1:1 to about 50:1. In preferred embodiments, the weight ratio of the solvent to the compound comprising Formula (II) may range from about 1:1 to about 5:1, from about 5:1 to about 10:1, from about 10:1 to about 15:1, from about 15:1 about 25:1, or from about 15:1 to about 50:1. In exemplary embodiments, the weight ratio of the solvent to the compound comprising Formula (II) may range from about 10:1 to about 20:1, or more preferably about 12:1.

### (d) Step B—Reaction Conditions

Typically, Step B of the process of the invention is allowed to proceed at a temperature that may range from about 0° to about 200° C. In preferred embodiments, the temperature of the reaction may range from about 50° to about 100° C., or from about 100° to about 150° C. In exemplary embodiments, the temperature of the reaction may range from about 80° to about 120° C. In one exemplary embodiment, the temperature of the reaction may range from about 80° to about 100° C. In another exemplary embodiment, the temperature of the reaction may range from about 100° to about 120° C. The reaction may be conducted under ambient pressure, and preferably under an inert atmosphere (e.g., nitrogen or argon).

The duration of the reaction can and will vary. For example, the reaction may be allowed to proceed for about 1 hr, 2, hr, 3, hr, 4, hr, 5, hr, 6 hr, 8 hi, 10 hr, 12 hr, 15 hr, 18 hr, or 24 hr. Typically, however, the reaction is allowed to proceed for a sufficient period of time until the reaction is complete, as determined by any method known to one skilled in the art, such as chromatography (e.g., HPLC). In this context, a “completed reaction” generally means that the reaction mixture contains a significantly diminished amount of the compound comprising Formula (II) and a significantly increased amount of the compound comprising Formula (III) compared to the amounts of each present at the beginning of the reaction. Typically, the amount of the compound comprising Formula (II) remaining in the reaction mixture may be less than about 3%, less than about 1%, and preferably less than about 0.5%.

Upon completion of the reaction, the reaction mixture may be cooled and the product may be isolated by distillation, phase extraction, precipitation, filtration, crystallization, or other means familiar to those of skill in the art. The final product may be washed and dried, and analyzed by HPLC, HPLC, MS, NMR, IR, or TGA.

The yield of the compound comprising Formula (III) can and will vary. Typically, the molar yield of the compound comprising Formula (III) may be at least about 60%. In preferred embodiments of the invention, the molar yield of the compound comprising Formula (III) may be at least about 65%, or at least about 70%. In exemplary embodiments, the molar yield of the compound comprising Formula (III) may be at least about 75%, at least about 80%, or at least about 85%. In other exemplary embodiments, the molar yield of the compound comprising Formula (IH) may be at least about 90%, at least about 95%, at least 97%, or at least about 99%.

The compound comprising Formula (III) prepared by the process of the invention may be an end product itself, or may be further derivatized in one or more steps to yield further intermediates or end products. As an example, the compound comprising Formula (III) may be converted into a pharmaceutically acceptable salt using techniques well known to those of skill in the art.

### (e) Exemplary Embodiment

In an exemplary embodiment, a compound comprising Formula (Ia) is contacted with a transition metal catalyst in the presence of a protic solvent and at a temperature ranging from 65° to about 100° C. to form a compound comprising Formula (IIa). The compound comprising Formula (IIa) then is contacted with a methylating agent and a proton acceptor in the presence of an organic solvent and at a temperature ranging from 50° to about 150° C. to form a compound comprising Formula (IIIa). For the

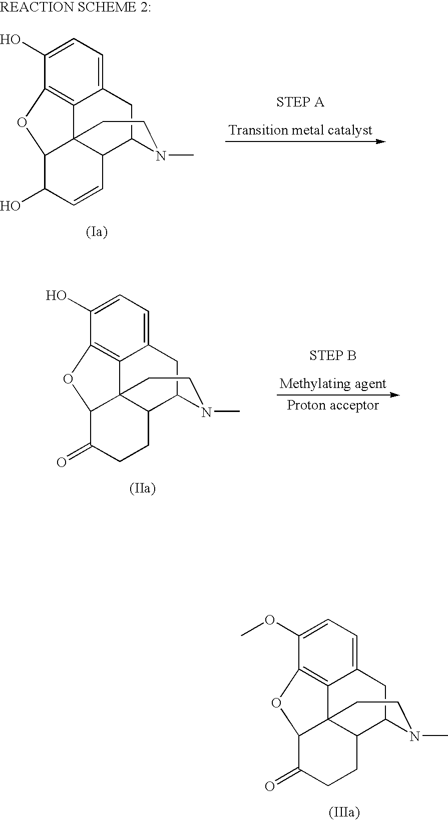

purpose of illustration, Reaction Scheme 2 depicts this aspect of the invention:

In one embodiment, the weight ratio of the compound comprising Formula (Ia) to the transition metal catalyst is from about 1:0.005 to about 1:0.02. In another embodiment, the molar ratio of the compound comprising Formula (IIa) to the methylating agent to the proton acceptor is from about 1:0.8:5 to about 1:2:10.

In exemplary embodiments, the transition metal catalyst may be RuCl₃, RhCl(PPh₃)₃, or Ru black; the methylating agent may be trimethylphenylammonium chloride, and the proton acceptor may be sodium ethoxide or sodium hydroxide.

(f) stereochemistry

The substrates and the products of the processes of the invention are morphinan compounds. For the purposes of discussion, the ring atoms of a morphinan compound are numbered as diagrammed below. Morphinan compounds have stereogenic atoms. In particular, the core morphinan

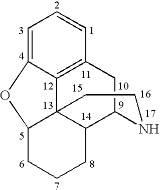

compound may have at least four chiral carbons; namely, C-5, C-13, C-14, and C-9.

Any of the compounds comprising Formulas (II), (IIa), (III), or (IIIa) may have a (−) or (+) orientation with respect to the rotation of polarized light, depending upon whether the starting substrate has (−) or (+) optical activity. More specifically, each chiral atom has an R or an S configuration. In particular, the configuration of the stereogenic carbons C-5, C-13, C-14, and C-9 may be RRRR, RRSR, RRRS, RRSS, RSRR, RSSR, RSRS, RSSS, SRRR, SRSR, SRRS, SRSS, SSRR, SSSR, SSRS, or SSSS, provided that the C-15 and the C-16 carbons are both either on the alpha face or the beta face of the molecule.

Additionally, any of the compounds comprising Formulas (II), (IIa), (III), or (IIIa) may be a pharmaceutically acceptable salt. Suitable pharmaceutically acceptable salts include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methanesulfonate, monohydrate, mutate, nitrate, oxalate, phenylpropionate, phosphate, phthalate, propionate, pyruvate, salicylate, stearate, succinate, sulfate, tannate, tartrate, terephthalate, valerate, and the like.

In a preferred embodiment, the compound comprising Formula (IIIa) produced by the process of the invention is a compound as diagrammed below. In one exemplary embodiment, the optical activity of the compound may be (+), and the configuration of C-5, C-13, C-14, and C-9, respectively, may be SRSS. In another exemplary embodiment, the optical activity of the compound may be (−), and the configuration of C-5, C-13, C-14, and C-9, respectively, may be RSRR.

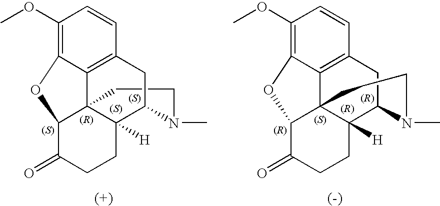

### DEFINITIONS

The compounds described herein have stereogenic atoms. Compounds of the present invention containing stereogenic centers may be isolated in optically active or racemic form. All chiral, diastereomeric, racemic forms and all geometric isomeric forms of a structure are intended, unless the specific stereochemistry or isomeric form is specifically indicated.

The term “acyl,” as used herein alone or as part of another group, denotes the moiety formed by removal of the hydroxy group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “allyl,” as used herein not only refers to compound containing the simple allyl group (CH₂═CH—CH₂—), but also to compounds that contain substituted allyl groups or allyl groups forming part of a ring system.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl (Ph), biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo” as used herein alone or as part of another group refer to chloride, bromide, fluoride, and iodide. The term “halide” as used herein refers to a binary compound comprising a halogen atom.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples illustrate various embodiments of the invention.

### Example 1

### Methylation of Hydromorphone to Hydrocodone—Trial 1

To a mixture of hydromorphone base (2.0 g, 7.01 mmol) and toluene (22.6 mL, 11.3 mL/g hydromorphone base) was added a mixture consisting of 21% sodium ethoxide (2.24 g, 6.91 mmol, 1.12 g/g hydromorphone base), ethanol (2.80 mL, 1.40 mL/g hydromorphone base), and phenyltrimethylammonium chloride (1.16 g, 6.76 mmol, 0.58 g/g hydromorphone base) at room temperature. The resulting mixture was heated to 60° C. and stirred 17 h. Deionized water (12.0 g, 6.0 g/g hydromorphone base) was added to the reaction mixture, the pH was adjusted to 11.5 with 50% NaOH, stirred 5 minutes, the layers were separated, and the aqueous layer was discarded. Deionized water (20.0 g, 10.0 g/g hydromorphone base) was added to the organic layer, the pH was adjusted to 5.1 with 98% H₂SO₄, stirred for 5 min, the layers were separated, and the organic layer was discarded, The pH of the aqueous layer was adjusted to 9.5 with concentrated ammonia, the slurry was stirred 15 minutes, filtered, washed with deionized water (10.0 g, 5.0 g/g hydromorphone base), and dried at 60° C. to afford 1.08 g (3.61 mmol, 51.5% non-assay adjusted yield) to a tan-colored solid.

### Example 2

### Methylation of Hydromorphone to Hydrocodone—Trial 2

Hydromorphone base (10.0 g, 35.1 mmol) and toluene (113 mL, 11.3 mUg hydromorphone base) were mixed in a 250 mL three-necked, jacketed round bottomed flask (set point 25.0° C.) equipped with a reflux condenser (set point 10° C.), thermocouple, polished glass stirring shaft, nitrogen inlet, and silicone oil bubbler. To this slurry was added a mixture consisting of 21% sodium ethoxide (11.6 g, 25.8 mmol, 1.16 g/g hydromorphone base), ethanol (14.0 mL, 1.40 ml/g hydromorphone base), and phenyltrimethylammonium chloride (6.20 g, 36.1 mmol, 0.620 g/g hydromorphone base) over a 5 min period at room temperature. The resulting mixture was heated to 80° C., stirred 2 h, and cooled to 55° C. The flask was then equipped with a short-path distillation head, laboratory house vacuum was applied, and ethanol was distilled from the reaction mixture. Toluene (52 mL, 5.2 mL/g hydromorphone base) and deionized water (60.0 g, 6.00 g/g hydromorphone base) were added and the pH was adjusted to approximately 11.0-11.1 with 50% NaOH (0.190 g, 0.019 g/g hydromorphone base). The layers were separated, deionized water (100.0 g, 10.0 g/g hydromorphone base) was added, the mixture was reheated to 60° C., and 98% H₂SO₄ (1.1 g, 11.0 mmol, 0.11 g/g hydromorphone base) was added. The mixture was cooled to 25° C. and 98% H₂SO₄ (1.60 g, 16.0 mmol, 0.16 g/g hydromorphone base) was added to adjust the pH to 5.5. The mixture was stirred for 10 min, the layers were separated, and the toluene layer was discarded. The pH was adjusted to 11.6 with 50% NaOH (2.42 g, 30.3 mmol, 0.24 g/g hydromorphone base), stirred 30 min, filtered, washed with deionized water (100 mL, 10.0 g/g hydromorphone base), and dried in a forced air oven at 60° C. to a constant weight to afford 8.42 g at 88.44% w/w (7.45 g at 100% w/w, 24.9 mmol, 70.9%) of hydrocodone alkaloid as an off-white solid.

### Example 3

### Synthesis of Hydrocodone from Morphine—Trial

Methanol (50.0 mL, 39.5 g, 7.90 g/g morphine alkaloid) was charged to a 250 mL three-necked, jacketed round bottomed flask (set point 70.0° C.) equipped with a reflux condenser (set point 10° C.), thermocouple, polished glass stirring shaft, nitrogen inlet, and silicone oil bubbler. At 60° C., morphine alkaloid (5.0 g, 17.5 mmol) was added and a solution was observed. Wilkinson's catalyst (RhCl(PPh₃)₃, 0.065 g, 0.070 mmol, 0.4 mol %, 0.013 g/g morphine alkaloid) was added and the resulting mixture was stirred 1 h at reflux. Toluene (56.5 mL, 48.9 g, 9.78 g/g morphine alkaloid) was added and the flask was equipped with a short-path distillation head. The jacket temperature was increased to 75° C. and a mixture of methanol and toluene (47.6 g) was distilled from the reaction mixture at atmospheric pressure. Toluene (7.0 mL, 8.1 g, 1.6 g/g morphine alkaloid) was added and the mixture was cooled to 30-35° C. A mixture consisting of 21% sodium ethoxide (5.80 g, 17.9 mmol, 1.16 g/g morphine alkaloid), ethanol (7.0 mL, 1.40 mL/g morphine alkaloid), and phenyltrimethylammonium chloride (3.10 g, 18.1 mmol, 0.620 g/g morphine alkaloid) was added over a 5 min period. The resulting mixture was heated to 80° C., stirred 5 h, cooled to 30° C., and stirred overnight. The flask was equipped with a short-path distillation head, the mixture was heated to 60° C., laboratory house vacuum was applied, and ethanol was distilled from the reaction mixture. Toluene (56.5 mL, 48.9 g, 9.78 g/g morphine alkaloid) and deionized water (30.0 g, 6.00 gig morphine alkaloid) were added and the pH was adjusted to approximately 11.0 with 50% NaOH (0.172 g, 0.0344 g/g morphine alkaloid) and stirred 15 min. The layers were separated (the aqueous layer was discarded), deionized water (50.0 g, 10.0 g/g morphine alkaloid) was added, the mixture was reheated to 60° C., and 98% H₂SO₄ (0.55 g, 5.6 mmol, 0.11 g/g morphine alkaloid) was added. The mixture was cooled to 25-30° C. and 98% H₂SO₄ (0.491 g, 4.91 mmol, 0.0982 g/g morphine alkaloid) was added to adjust the pH to 5.1. The mixture was stirred for 10 min, the layers were separated, and the toluene layer was discarded. The pH was adjusted to 12.4 with 50% NaOH (1.38 g, 17.3 mmol, 0.276 g/g morphine alkaloid), stirred 45 min, filtered, washed with deionized water (25.0 g, 5.00 g/g morphine alkaloid), and dried in a forced air oven at 55° C. to a constant weight to afford 3.88 g (13.0 mmol, 74.3%, non assay adjusted molar yield) of hydrocodone alkaloid as an off-white solid

### Example 4

### Synthesis of Hydrocodone from Morphine—Trial 2

Ethanol (75.0 mL, 59.2 g, 2.14 g/g morphine alkaloid), deionized water (75.0 g, 2.14 g/g morphine alkaloid), and morphine alkaloid (35.0 g as is, 26.3 g @100%, 92.2 mmol) was charged to a 250 mL three-necked, jacketed round bottomed flask equipped with a reflux condenser (set point 10° C.), thermocouple, polished glass stirring shaft, nitrogen inlet, and silicone oil bubbler. With stirring, 98% H₂SO₄ (9.55 g, 0.273 g/g morphine alkaloid) was added and a solution was observed. Wet ruthenium black (1.27 g as is, 0.865 g@100%, 0.0247 g/g morphine alkaloid) was added and the mixture was stirred for 5.5 h at reflux. The flask was equipped with a short-path distillation head and an ethanol/water mixture (75 ml) was distilled from the reaction mixture at atmospheric pressure. The mixture was cooled to 60-65° C. and the catalyst was removed by filtration. The pH was adjusted to 9.0 with 28% NH₃ (43.2 g, 1.23 g/g morphine alkaloid) and the resulting slurry was cooled to room temperature, filtered, and the resulting off-white solid was washed with deionized water (50.0 g, 1.43 g morphine alkaloid) and dried at 60° C. in a forced air oven to afford 18.8 g (65.9 mmol, 71.5%, non assay adjusted molar yield) of hydromorphone base as an off white solid.

The hydromorphone base (18.8 g, 65.9 mmol, non assay adjusted) was mixed with toluene (212 mL, 11.3 mL/g hydromorphone base) in a 500 mL three necked jacketed round bottomed flask (set point 25.0° C.) equipped with a reflux condenser (set point 10° C.), thermocouple, polished glass stirring shaft, nitrogen inlet, and silicone oil bubbler. To this slurry was added a mixture consisting of 21% sodium ethoxide (21.8 g, 1.16 g/g hydromorphone base), 2B alcohol (26.3 mL, 1.40 mL/g hydromorphone base), and phenyltrimethylammonium chloride (11.7 g, 0.62 g/g hydromorphone base) over a 5 min period at room temperature. The resulting mixture was heated to 80 DC, stirred for 3.5 h, and cooled to 60° C. The flask was then equipped with a short-path distillation head, laboratory house vacuum was applied, and ethanol was distilled from the reaction mixture. Toluene (98 mL, 5.2 mL/g hydromorphone base) and deionized water (113 g, 6.0 g/g hydromorphone base) were added and the pH was adjusted to 11.0-11.5 with 50% NaOH (0.494 g, 0.0263 g/g hydromorphone base). The layers were separated, deionized water (188 g, 10.0 g/g hydromorphone base) was added, the mixture was reheated to 60° C., and 98% H₂SO₄ (2.1 g, 0.11 g/g hydromorphone base) was added. The mixture was cooled to 25° C. and 98% H₂SO₄ (1.5 g, 0.080 g/g hydromorphone base) was added to adjust the pH to 5.3. The mixture was stirred for 10 min, the layers were separated, and the toluene layer was discarded. The pH was adjusted to 11.7 with 50% NaOH (5.23 g, 0.278 g/g hydromorphone base), stirred 30 min, filtered, washed with deionized water (188 g, 10.0 g/g hydromorphone base), and dried in a forced air oven at 60° C. to a constant weight to afford 17.5 g (58.5 mmol, 63.5%) of hydrocodone alkaloid as an off-white solid.

What is claimed is:

1. A process for the preparation of a 6-keto, 3-alkoxy morphinan, the process comprising (a) contacting a 6-hydroxy, 3-hydroxy morphinan with a transition metal catalyst to form a 6-keto, 3-hydroxy morphinan; and (b) contacting the 6-keto, 3-hydroxy morphinan with an alkylating agent and a proton acceptor to form the 6-keto, 3-alkoxy morphinan.

2. The process of claim 1, wherein the transition metal catalyst comprises a transition metal chosen from ruthenium, rhodium, palladium, osmium, iridium, and platinum; the transition metal catalyst is chosen from a transition metal element, a transition metal salt, and a transition metal complex; the alkylating agent comprises a hydrocarbyl or substituted hydrocarbyl group and is chosen from halide, oxide, sulfonate, carbonate, and quaternary ammonium salt; and the proton acceptor has a pKb less than about 6.

3. A process for the preparation of a compound comprising Formula (III), the process comprising:
(a) contacting a compound comprising Formula (I) with a transition metal catalyst to form the compound comprising Formula (II); and
(b) contacting the compound comprising Formula (II) with an alkylating agent comprising R³ and a proton acceptor to form the compound comprising Formula (III): 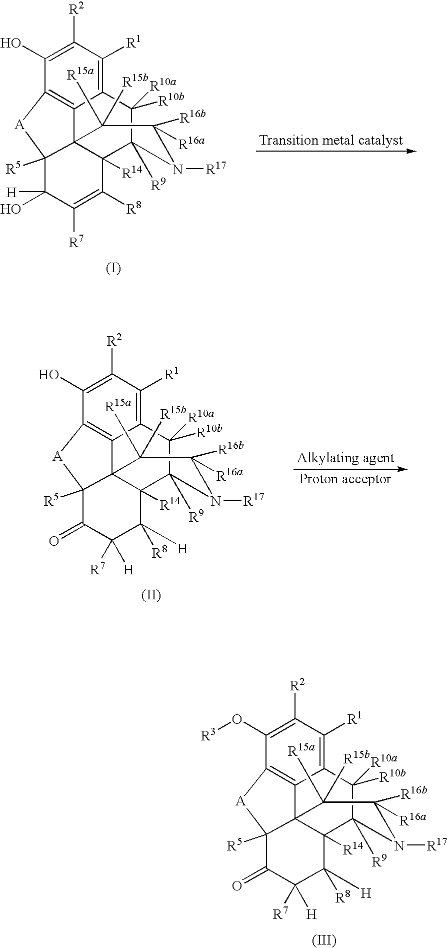
wherein:
A is a heteroatom selected the group consisting of oxygen and sulfur;
R¹ and R² are independently chosen from hydrogen, halogen, hydroxy, protected hydroxy,
{—}SH, {—}SR¹⁶¹¹, {—}OR¹⁶¹¹, and {—}NR¹⁶¹¹R¹⁶¹², hydrocarbyl, and substituted hydrocarbyl;
R³ is chosen from hydrocarbyl and substituted hydrocarbyl;
R⁵, R⁷, R⁸, R⁹, R^{10a}, R^{10b}, R¹⁴, R^{15a}, R^{15b}, R^{16a}, R^{16b}, and R¹⁷ are independently chosen from hydrogen, halogen, hydroxy, {—}SH, {—}SR¹⁶¹¹, {—}OR¹⁶¹¹, and {—}NR¹⁶¹¹R¹⁶¹², hydrocarbyl, and substituted hydrocarbyl; provided that any of R^{10a} and R^{10b}, R^{15a} and R^{15b}, and R^{16a} and R^{16b} may together form a moiety chosen from {═}O, {═}S, and {═}NR¹⁶¹³;
R¹⁶¹¹, R¹⁶¹², and R¹⁶¹³ are independently chosen from hydrocarbyl, and substituted hydrocarbyl; and
one or more of R¹, R², R³, R⁵, R⁷, R⁵, R⁹, R^{10a}, R^{10b}, R¹⁴, R^{15a}, R^{15b}, R^{16a} and R^{16b} may form part of a ring or ring system chosen from carbocyclic, heterocyclic, aryl, heteroaryl, and combinations thereof.

4. The process of claim 3, wherein A is oxygen; R¹, R², R⁵, R⁷, R⁸, R⁹, R^{10a}, R^{10b}, R^{15a}, R^{15b}, R^{16a}, and R^{16b} are hydrogen; R³ is chosen from alkyl, substituted alkyl, hydroxyalkyl, carboxyalkyl, (alkyloxycarbonyl)alkyl, aryl alkyl, alkenyl, substituted alkenyl, aryl, and substituted aryl; R¹⁴ is hydrogen or hydroxy; and R¹⁷ is chosen from hydrogen, alkyl, cycloalkyl, cycloalkylmethyl, allyl, and aryl.

5. The process of claim 4, wherein R³ is methyl; R¹⁴ is hydrogen; and R¹⁷ is methyl.

6. The process of claim 3, wherein the transition metal catalyst comprises a transition metal chosen from ruthenium, rhodium, palladium, osmium, iridium, and platinum.

7. The process of claim 3, wherein the transition metal catalyst is chosen from a transition metal element, a transition metal salt, and a transition metal complex.

8. The process of claim 7, wherein the transition metal element is chosen from ruthenium black and palladium black.

9. The process of claim 7, wherein the transition metal salt is chosen from RuCl₃, PdCl₂, and Pd(OAc)₂.

10. The process of claim 7, wherein the transition metal complex is chosen from RuCl₂(PPb₃)₃, RuCl₂(PPh₃)₄, RuH₂(PPh₃)₄, RhCl(PPh₃)₃, RuCl₂(dmso)₄, and a bis-allyl ruthenium complex.

11. The process of claim 3, wherein step (a) is conducted in the presence of a solvent; the weight ratio of the solvent to the compound comprising Formula (I) is from about 0.5:1 to about 10:1; the solvent is chosen from a protic solvent, an aprotic solvent, and combinations thereof; and the weight ratio of the compound comprising Formula (I) to the transition metal catalyst is from about 1:0.0001 to about 1:0.1.

12. The process of claim 3, wherein step (a) is conducted at a temperature from about 10° C. to about 120° C.

13. The process of claim 3, wherein the alkylating agent is chosen from halide, oxide, sulfonate, carbonate, and quaternary ammonium salt.

14. The process of claim 3, wherein the proton acceptor has a pKb less than about 6 and is chosen from an alkali or alkaline earth metal hydroxide; an alkali or alkaline earth metal carbonate; an alkali or alkaline earth metal bicarbonate; an alkali metal alkoxide or aryloxide; an alkali metal alkylcarboxylate or arylcarboxylate; an alkali metal phosphate; an alkali or alkaline earth metal amide, a tertiary amine; and combinations thereof.

15. The process of claim 3, wherein step (b) is conducted in the presence of a solvent; the weight ratio of the solvent to the compound comprising Formula (II) is from about 1:1 to about 50:1; the solvent is chosen from an organic solvent, an aprotic solvent, a protic solvent, and combinations thereof; the reaction is conducted at a temperature of about 0° C. to about 200° C.; and the molar ratio of the compound comprising Formula (II) to the alkylating agent to the proton donor is from about 1:0.5:1 to about 1:5:20.

16. The process of claim 3, wherein the weight ratio of the compound comprising Formula (I) to the transition metal catalyst is from about 1:0.0001 to about 1:0.1; step (a) is conducted in the presence of a protic solvent and at a temperature from about 10° to about 120° C.; the molar ratio of the compound comprising Formula (II) to the alkylating agent to the proton acceptor is from about 1:0.5:1 to about 1:5:20; the proton acceptor has a pKb less than about 6; and step (b) is conducted in the presence of an organic solvent and at a temperature from about 0° to about 200° C.

17. The process of claim 3, wherein the optical activity of the compounds comprising Formulas (I), (II), and (III) is (−) or (+), and the configuration of C-5, C-13, C-14, and C-9, respectively, is chosen from RRRR, RRSR, RRRS, RRSS, RSRR, RSSR, RSRS, RSSS, SRRR, SRSR, SRRS, SRSS, SSRR, SSSR, SSRS, and SSSS, provided that the C-15 and the C-16 carbons are both either on the alpha face of the molecule or the beta face of the molecule.

18. The process of claim 5, wherein the transition metal catalyst is RuCl₃, (RhCl(PPh₃)₃, or Ru black; the weight ratio of the compound comprising Formula (I) to the transition metal catalyst is from about 1:0.005 to about 1:0.02; step (a) is conducted in the presence of a protic solvent, and at a temperature from about 65° to about 100° C.; the alkylating agent is trimethylphenylammonium halide; the proton acceptor is sodium ethoxide or sodium hydroxide; the molar ratio of the compound comprising Formula (II) to the alkylating agent to the proton acceptor is from about 1:0.8:5 to about 1:2:10; and step (b) is conducted in the presence of an organic solvent and at a temperature from about 50° to about 150° C.

19. The process of claim 18, wherein the optical activity of the compounds comprising Formulas (I), (II), and (III) is (−), and the configuration of C-5, C-13, C14, and C-9, respectively, is RSRR.

20. The process of claim 18, wherein the optical activity of the compounds comprising Formula (I), (II), and (III) is (+), and the configuration of C-5, C-13, C-14, and C-9, respectively, is SRSS.
